(19) United States
(12) Patent Application Publication
(10) Pub. No.: US 2004/0126340 A1
(43) Pub. Date: Jul. 1, 2004

(54) Ultraviolet-screening and color-chaning composition and its preparing method

(76) Inventor: Kyong-Rae Jo, Daegu-city (KR)

Correspondence Address: Paul J Farrell, Dilworth & Barrese, 333 Earle Ovington Boulevard, Uniondale, NY, 11553, US

(21) Appl. No.: 10/477,485

(22) PCT Filed: Mar. 20, 2002
(86) PCT No.: PCT/KR02/00467

(30) Foreign Application Priority Data
May 12, 2001 (KR) 2001-0025998

(51) Int. Cl. A61K 7/42

(52) U.S. Cl. 424/59

(57) ABSTRACT

Disclosed is a UV-screening and color-changing composition and its preparing method, the composition comprising spiro oxazine and spiro pyran as an optical color-changing pigment combined with an animal fat or vegetable oil, and additionally an antioxidant and a stabilizer, which composition has a higher sun protection factor (SPF) and can be used as a cosmetic or skin-protecting photo-sensitive material to increase the color-developing concentration when exposed to solar or UV radiation and to provide a cream or oil formation ready to color development or decolorization. The composition has a higher SPF number compared with the conventional skin protecting compositions and, particularly has an ability of changing in color and screening UV radiation without any irritation on the skin.

### TECHNICAL FIELD

The present invention relates to a composition capable of screening ultraviolet (UV) rays and changing the color of a UV-screening material applied on the skin by the UV rays. More particularly, the present invention relates to a UV-screening and color-changing composition and its preparing method, the composition comprising spiro oxazine and spiro pyran as an photorome powder combined with an animal/vegetable oil, and additionally an antioxidant and a stabilizer, which composition has a higher sun protection factor (SPF) and can be used as a cosmetic or skin-protecting photo-sensitive material to increase the color development concentration when exposed to solar or UV radiation and to provide a cream or oil formation ready to color development or decolorization.

### BACKGROUND ART

In general, many people understand that over-exposure to solar radiation can sunburn, skin damage and an increased risk of developing skin cancer as well as photo augmented skin aging and that an appropriate sun protection material should be applied to the skin for outdoor activity.

Recently, there is an increasing interest in the problems with sunscreen products that have a high SPF value and contain an organic sunscreen agent, such as irritation and susceptibility that may occur in some users of such sunscreen products. Sun protection factor (SPF) is a measure of the effectiveness of a sunscreen product.

The SPF value is determined as a ratio of the exposed amount of the sunscreen product necessary to cause a minimum number of red spots on the protected skin to the exposed amount of the product necessary to cause a minimum number of red spots on an unprotected skin.

Ultraviolet radiations are divided into ultraviolet A, ultraviolet B and ultraviolet C. Especially, ultraviolet A has a longest wavelength that penetrates into the deepest part of the skin (inner skin) and causes skin aging such as wrinkles.

Ultraviolet A also causes pigmentation and participates in sun tanning in summer.

Ultraviolet B has an intermediate wavelength of 290 to 320 nm that affects the outermost layer of the skin and increases cell division to make the horny layer of the skin and to cause freckles.

Ultraviolet C has a shortest wavelength and is the most dangerous ultraviolet that may damage the skin to cause skin cancer but does not arrive at the surface of the earth because it is absorbed by the ozone layer in the atmosphere.

In particular, over-exposure of the ultraviolet C having an intermediate wavelength of 290 to 320 nm (1 nm=10⁻⁹ m) called UVB may cause acute dermatitis with irritations such as sharp pain, dropsical swelling and blister, and on occasions, irritations over the whole body, such as shock, rigor, fever or nausea.

Apparent symptoms that appear within about 6 to 12 hours of exposure to the ultraviolet radiation reach their peak in 24 to 28 hours and then slowly mitigates. Depending on the severity of sunburn, the affected part of the skin gets flaked off.

Some people who desire perfect skin protection from ultraviolet radiation may apply a sunscreen product of an extremely high SPF number. But, an over-use of the UV-screening products that are substantially harmless and safe to the skin may cause irritations on the skin, leading to cutaneous diseases such as basal-cell carcinoma (small and smooth nodes) normally appearing on the face in over-exposure to solar radiation.

To prevent such a damage on the skin, such a sunscreen product that contains para-aminobenzoic acid (PABA) or benzophenone as a UV absorber is used.

The UV absorber may absorb ultraviolet radiations to protect the skin from the radiations but readily penetrates into the skin and causes many side effects on the skin.

To solve this problem, Korean Patent Application No. 1997-709187 discloses a UV-screening composition that comprises an inorganic UV screening agent, an anionic emulsifier, a carrier oil, and an oil component containing at least one skin softener, the anionic emulsifier being selected from the group consisting of the salts of saturated fatty acids and linear fatty acids, alkyl sulfosuccinate, alkyl phosphate and mixtures thereof.

The composition comprises a plurality of UV-screening components in oil and needs application of oil after basic make-up so as to skin protection from UV radiations.

However, a color make-up after application of make-up base and oil combined with basic make-up may have a problem causing the cosmetics to come off the facial skin or making the face oily and deteriorate the UV screening efficiency.

### DISCLOSURE OF INVENTION

It is therefore an object of the present invention to prepare a UV-screening and color-changing cream comprising spiro oxazine as a safe principal component and to use the cream as a skin-protecting photo-sensitive material.

To achieve the object of the present invention, there is provided a UV-screening and color-changing composition and its preparing method, the composition comprising spiro oxazine as a principal component, and additionally an olive oil, an antioxidant and a stabilizer, which composition has a higher sun protection factor (SPF) and can be used as a cosmetic or skin-protecting photo-sensitive material to increase the color-developing concentration when exposed to solar or UV radiation and to provide a cream or oil formation ready to color development or decolorization.

### BRIEF DESCRIPTION OF THE DRAWINGS

Further objects and advantages of the invention can be more fully understood from the following detailed description taken in conjunction with the accompanying drawing in which:

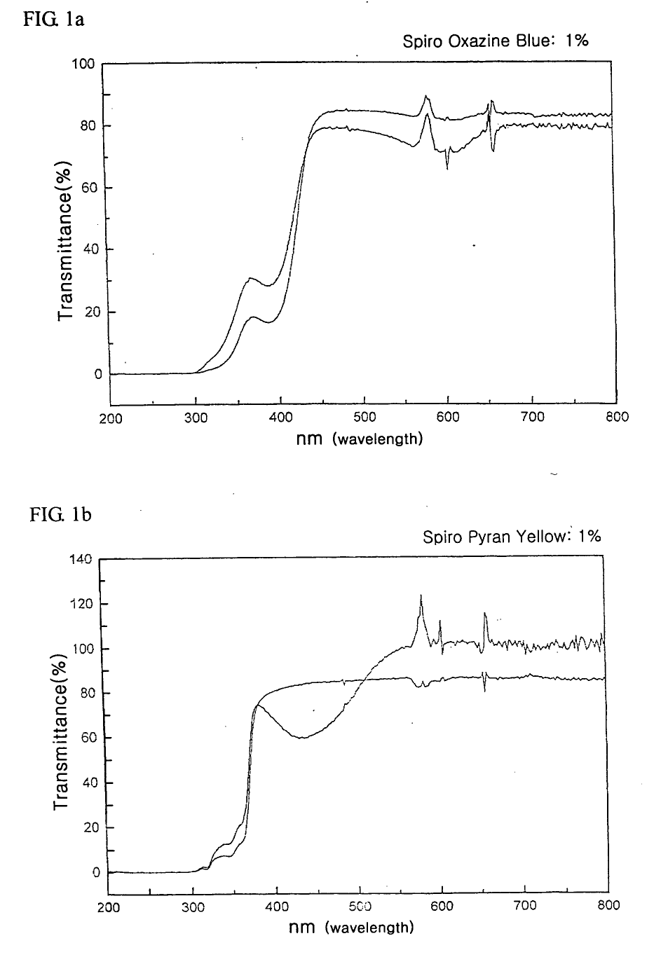

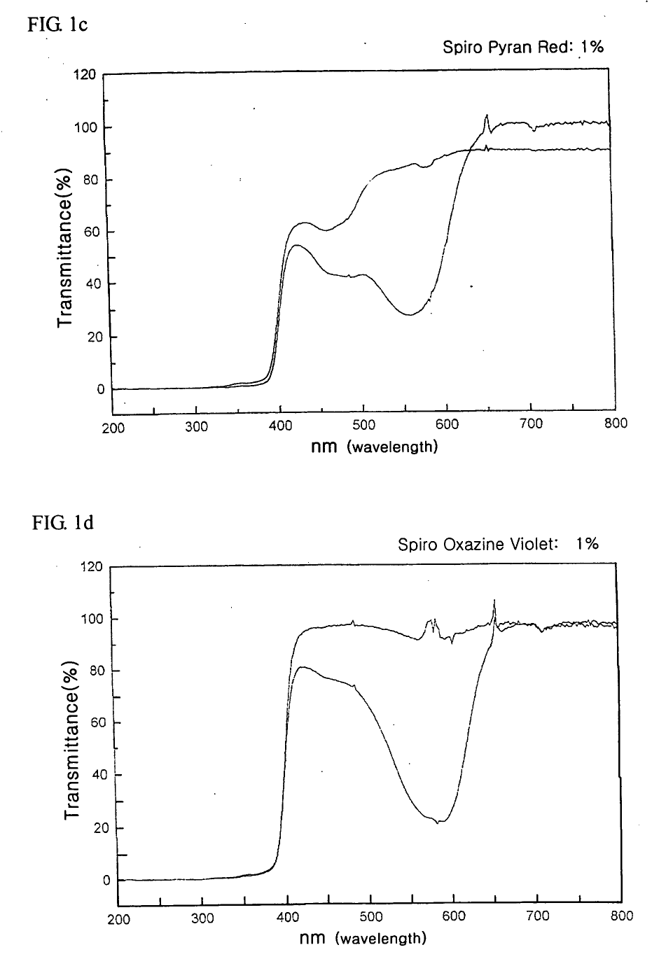

(a) to (d) of FIG. 1 are graphs showing that ultraviolet radiation is blocked by the composition according to an embodiment of the present invention; and

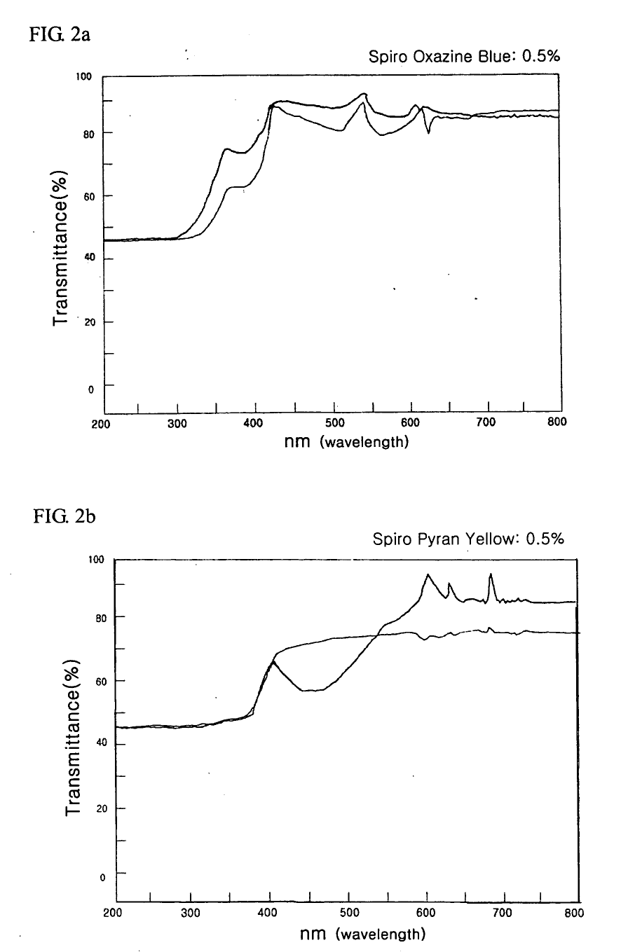

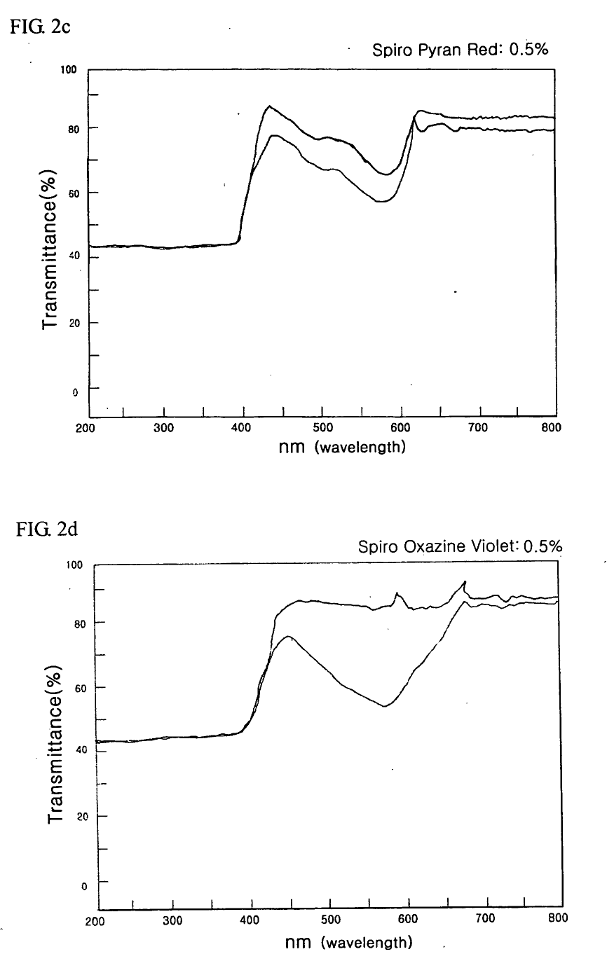

(a) to (d) of FIG. 2 are graphs showing that ultraviolet radiation is blocked by the composition according to another embodiment of the present invention.

### BEST MODE FOR CARRYING OUT THE INVENTION

The present invention comprises an olive oil, spiro oxazine (pyran) for changing the color of the composition, an antioxidant for regulating the lifetime and color development of the composition, and a stabilizer for maintaining the composition in the emulsified state.

Hereinafter, the preferred embodiments of the present invention will be described in detail with reference to the accompanying drawings.

(a) to (d) of FIG. 1 are graphs showing that UV radiation passes through a compound used as a component of the novel UV-screening composition of the present invention, and (a) to (d) of FIG. 2 are graphs showing that UV radiation passes through a compound used as a component of another novel UV-screening composition of the present invention.

The composition of the present invention contains at least the following components:

(A) Animal Fat or Vegetable Oil

Examples of the animal fat or vegetable oil as used herein may include glycerin, soybean oil, tung oil, lanoline, cotton seed oil, stearic acid, linseed oil, whale oil, hydrogenated oil, coconut oil, palm kermel oil, palm oil, oleic acid, lauric acid, myristic acid, behenic acid, palmitic acid, para-aminobenzoic acid (PABA), benzophenone, tallow, iso-stearic acid, fatty acid, rape seed oil, polyglycerin, castor-seed oil, castor oil, turkey red oil, vaseline, soybean oil and shotting oil.

(B) Spiro Oxazine (Pyran)

Spiro oxazine (pyran) is used as a principal component of the composition in the following content that makes the component have a higher color-developing concentration under sun or UV radiation and be susceptible to color development and decolorization under sun radiation and thereby permeable to a minimum UV radiation.

Spiro oxazine Blue: 1%

Spiro pyran yellow: 1%

Spiro pyran Red: 1%

Spiro oxazine Violet: 1%

(C) Antioxidant

Examples of the antioxidant as used herein for controlling lifetime, rancidity and color development of the composition may selectively include amines, phenols, aminophenols, normal tocopherol, propyl gallate, or butylated hydroxytoluene and hydroxyanizol. Especially, the most preferred antioxidant is Iganox 1010 and AO 30, which are highly efficient.

(D) Stabilizer

The stabilizer as used herein is closely related to an emulsifier that maintains the emulsified state and increases preservability to enhance storage stability, and can be derived from algae such as Hisorb-770, algin, carageenan and agar. Lecithin is also used as an emulsifier.

The ingredients as listed above are combined together in a defined amount and used as a sunscreen material applied on the skin.

Preferably, the composition applied to the skin is invisible and necessarily maximizes absorption of UV-B and UV-A radiations while reducing scattering of visible and ultraviolet radiations.

The UV-screening and color-changing composition having a high SPF number according to the present invention can be prepared by mixing 1 g of spiro oxazine, 1 g of an oxidant and 2 g of a stabilizer with 100 g of an animal fat or vegetable oil, subjecting the mixture to heat treatment and stirring to prepared an aqueous solution, and causing precipitation in the aqueous solution.

The animal fat or vegetable oil (A) may be chosen depending on the type of cosmetics or body-protecting products, such as body lotion, hand lotion, oil, ointment, lipstick, facial cosmetics or the like.

Now, the present invention will be described in detail by way of the following examples. The compositions prepared in the examples were measured in regard to the SPF number with the SPF system.

### EXAMPLE 1

1 g of spiro oxazine Blue (1%), 1 g of an antioxidant (hydroxytoluene and hydroxyanizol) and 2 g of a stabilizer (Lecithin) were mixed with each 100 g of glycerin, soybean oil, tung oil and Vaseline at 95° C. with stirring. The resulting aqueous solution was then subjected to precipitation and cooling.

The composition thus obtained in Example 1 had a SPF number of 34 and was excellent in properties.

TABLE 1

| Ingredient | Wt. % |
|---|---|
| Glycerin | 58.4 |
| Spiro oxazine Blue | 12.5 |
| Antioxidant | 16.6 |
| Stabilizer | 12.5 |
| | 100.0 |

* Structural formula of Spiro oxazine Blue

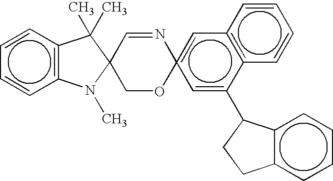

### EXAMPLE 2

1 g of spiro pyran Yellow (1%), 1 g of an antioxidant (aminophenols or amines) and 2 g of a stabilizer (Lecithin) were mixed with each 100 g of lanoline, palmitic acid and iso-stearic acid at about 90° C. with stirring. The resulting aqueous solution was then subjected to precipitation and cooling.

The composition thus obtained in Example 2 had a SPF number of 37 and was excellent in properties.

TABLE 2

| Ingredient | Wt. % |
|---|---|
| Lanoline | 52.2 |
| Spiro pyran Yellow | 12.5 |
| Antioxidant | 13.8 |
| Stabilizer | 21.5 |
| | 100.0 |

* Structural formula of spiro pyran Yellow

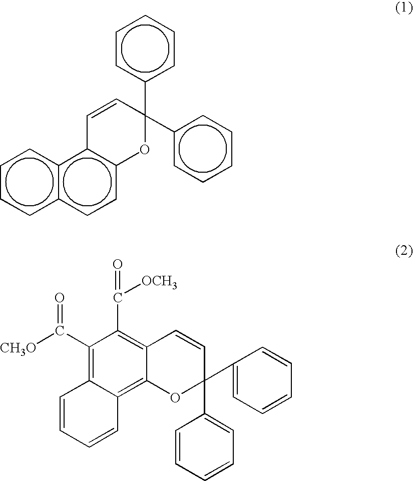

### EXAMPLE 3

1 g of spiro pyran Red (1%), 1 g of an antioxidant (hydroxytoluene and amines) and 2 g of a stabilizer (Lecithin) were mixed with each 100 g of stearic acid, fatty acid, oleic acid, lauric acid, myristic acid and behenic acid at about 90° C. with stirring. The resulting aqueous solution was then subjected to precipitation and cooling. The composition thus obtained in Example 3 had a SPF number of 32 and was excellent in properties.

TABLE 3

| Ingredient | Wt. % |
|---|---|
| Stearic acid | 54.8 |
| Spiro pyran Red | 11.2 |
| Antioxidant | 13.3 |
| Stabilizer | 20.7 |
| | 100.0 |

* Structural formula of spiro pyran Red

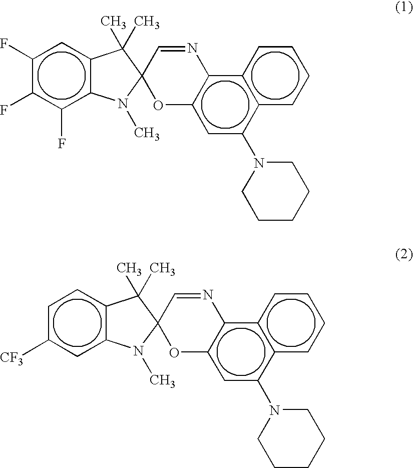

### EXAMPLE 4

1 g of spiro oxazine Violet (1%), 1 g of an antioxidant (hydroxyanizol) and 2 g of a stabilizer (emulsifier) were mixed with each 100 g of linseed oil, whale oil, hydrogenated oil, coconut oil and palm kennel oil at about 95° C. with stirring. The resulting aqueous solution was then subjected to precipitation and cooling.

The composition thus obtained in Example 4 had a SPF number of 35 and excellent in properties.

TABLE 4

| Ingredient | Wt. % |
|---|---|
| Linseed oil | 55.7 |
| Spiro oxazine Violet | 11.9 |
| Antioxidant | 12.8 |
| Stabilizer | 19.6 |
| | 100.0 |

* Structural formula of spiro oxazine Violet

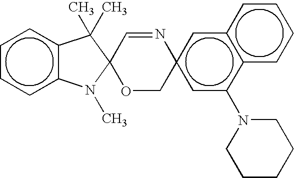

According to the examples, the UV-screening and color-changing compositions thus obtained had a satisfactory SPF number.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

### INDUSTRIAL APPLICABILITY

As described above, the novel UV-screening and color-changing composition of the present invention has a higher SPF number compared with the conventional skin protecting compositions and, particularly has an ability of changing in color and screening UV radiation without any irritation on the skin.

What is claimed is:

1. A UV-screening and color-changing composition, which is to protect skin and hairs from ultraviolet radiation, the composition comprising 1 part by weight of spiro oxazine as an optical color-changing pigment based on 100 parts by weight of an animal fat or vegetable oil, the composition being prepared by mixing the spiro oxazine with the animal fat or vegetable oil at 60 to 95° C. with stirring and subjecting the mixture to precipitation and cooling.

2. A method for preparing UV-screening and color-changing composition which is to protect skin and hairs from ultraviolet radiation, the method comprising the steps of:
mixing 1 part by weight of the spiro pyran as an optical color-changing pigment with 100 parts by weight of the animal fat at 60 to 95° C.;
stirring and subjecting the mixture to precipitation and cooling.

3. A UV-screening and color-changing composition, which is to protect skin and hairs from ultraviolet radiation, the composition comprising 1 part by weight of spiro oxazine as an optical color-changing pigment based on 100 parts by weight of an animal fat or vegetable oil, the composition being prepared by mixing spiro oxazine with the animal fat or vegetable oil at 90 to 95° C. with stirring and subjecting the mixture to precipitation and cooling.

4. A UV-screening and color-changing composition, which is to protect skin and hairs from ultraviolet radiation, the composition comprising 1 part by weight of spiro pyran as an optical color-changing pigment based on 100 parts by weight of an animal fat or vegetable oil, the composition being prepared by mixing the spiro pyran with the animal fat or vegetable oil at 60 to 95° C. with stirring and subjecting the mixture to precipitation and cooling.

5. The UV-screening and color-changing composition as claimed in any one of claims 1 to 4, further comprising 1 part by weight of an antioxidant selected from the group consisting of amines, phenols, aminophenols, normal tocopherol, propyl gallate, butylated hydroxytoluene and hydroxyanizol, Iganox 1010 and AO 30, the antioxidant being mixed with the mixture of the animal fat or vegetable oil and the optical color-changing pigment.

6. The UV-screening and color-changing composition as claimed in any one of claims 1 to 4, further comprising 2 parts by weight of a stabilizer selected from the group consisting of Hisorb-770, algin, carageenan and agar, the stabilizer being mixed with the mixture of the animal fat or vegetable oil and the optical color-changing pigment.

7. The UV-screening and color-changing composition as claimed in any one of claims 1 to 4, further comprising 1 part by weight of an antioxidant selected from the group consisting of amines, phenols, aminophenols, normal tocopherol, propyl gallate, butylated hydroxytoluene and hydroxyanizol, Iganox 1010 and AO 30, and 2 parts by weight of a stabilizer selected from the group consisting of Hisorb-770, algin, carageenan and agar, the antioxidant and the stabilizer being mixed with the mixture of the animal fat or vegetable oil and the optical color-changing pigment.

8. A UV-screening and color-changing composition comprising each. 0.2 to 10 parts by weight of spiro oxazine and spiro pyran based on 100 parts by weight of an animal fat or vegetable oil, the composition being prepared by mixing the spiro oxazine and the spiro pyran with the animal fat or vegetable oil at 60 to 90° C. with stirring and cooling the mixture into a cream or oil in different colors.
